(19) United States
(12) Patent Application Publication
(10) Pub. No.: US 2012/0150097 A1
(43) Pub. Date: Jun. 14, 2012

(54) MEDICAL INSTRUMENT FOR MICRO-INVASIVE APPLICATIONS

(75) Inventors: Daniel Neumueller, Weinheim (DE); Denis Reibel, Herrlisheim (FR); Dirk Grafahrend, Mannheim (DE)

(73) Assignee: CARL FREUDENBERG KG, Weinheim (DE)

(21) Appl. No.: 13/311,580

(22) Filed: Dec. 6, 2011

(30) Foreign Application Priority Data
Dec. 10, 2010 (EP) 10 015 519.1

(51) Int. Cl. A61M 31/00

(52) U.S. Cl. 604/15

(57) ABSTRACT

A medical instrument includes a tube, a bioresorbable nonwoven disposed in the tube, and a plunger disposed inside the tube that closes off a first end of the tube. The plunger is movable relative to the tube and slidable inside the tube so as to expel the nonwoven from the tube through actuation of the plunger.

### CROSS REFERENCE TO RELATED APPLICATIONS

This application claims priority to European Patent No. 10 015 519.1, filed Dec. 10, 2010, which is hereby incorporated by reference herein in its entirety.

### FIELD

The invention relates to a medical instrument.

### BACKGROUND

The trocar is a medical instrument often employed in minimally invasive surgical procedures. A trocar is used for a sharp or dull procedure within the scope of minimally invasive surgery in order to gain access to a body cavity (for example, the abdominal cavity or the thoracic cavity) which is then kept open by means of a cylindrical, tubular cannula.

Modern trocars are made of either titanium, surgical steel or plastic and are manufactured as either disposable or reusable instruments.

A tube with a plunger can be inserted into the trocar to serve as an administration or transportation means, and the tube and the plunger can be made of plastic.

Here, a plunger is accommodated in a tube having an inner diameter of about 0.5 mm to 12 mm, whereby the tip of the plunger closes off the opening of the tube. The tube and the plunger are located at least partially inside the trocar and are inserted through the trocar at least partially into the abdominal cavity through the abdominal wall. Materials that promote healing can be then be transported into the abdominal cavity through the tube and plunger.

After the plunger and/or tube has been removed, the surgeon can use an optical instrument to look into the abdominal cavity through the trocar or else to perform minimally invasive surgical procedures using grasping, cutting and other instruments.

Trocars are employed in laparoscopy, thoracoscopy and arthroscopy in order to examine body cavities or joint cavities.

In medical technology, there is often a need to introduce bioresorbable materials into the body of humans or animals in order to promote healing or to stanch bleeding.

In this context, it is desirable to bring the materials to the intended position without their being damaged. The bioresorbable materials employed so far are not sufficiently mechanically stable. Particularly when they are wet, the materials used up until now are not sufficiently stable.

### SUMMARY

In an embodiment, the present invention provides a medical instrument that is equipped in such a way that a bioresorbable material can be transported through a tube without being damaged and then easily positioned.

In an embodiment, the present invention provides a medical instrument including a tube, a bioresorbable nonwoven disposed in the tube, and a plunger disposed inside the tube that closes off a first end of the tube. The plunger is movable relative to the tube and slidable inside the tube so as to expel the nonwoven from the tube through actuation of the plunger.

### BRIEF DESCRIPTION OF THE DRAWINGS

Exemplary embodiments of the present invention are described in more detail below with reference to the drawings, in which:

FIG. 1 shows a sectional view of a trocar which is inserted into the abdominal wall, whereby the nonwoven is positioned inside a tube that is accommodated in the trocar;

FIG. 2 shows a sectional view of the trocar according to FIG. 1, which is inserted into the abdominal wall, whereby the nonwoven has been partially expelled from the tube; and

FIG. 3 shows a sectional view of the trocar according to FIGS. 1 and 2, which is inserted into the abdominal wall, whereby the nonwoven has been completely expelled from the tube.

### DETAILED DESCRIPTION

In an embodiment, the present invention provides a medical instrument including a tube and a plunger accommodated inside the tube, whereby the plunger can move relative to the tube and can be slid inside it, and whereby the plunger closes off a first end of the tube. A bioresorbable nonwoven is accommodated in the tube, whereby the nonwoven can be expelled from the tube by actuating the plunger. It has been surprisingly found that bioresorbable nonwovens can exhibit a very strong extensibility and stability and can also be pressed through tight cylindrical spaces inside a tube. Even though a person skilled in the art would have expected that filigree structures such as bioresorbable nonwovens would tear when they are pushed through a tube, this path was successfully traversed according to the invention.

Particularly surprisingly, it was found that bioresorbable nonwovens remain sufficiently stable even they are wet or moist, especially after being soaked with bodily fluids. This yields a medical instrument that is equipped in such a way that a bioresorbable material can be transported through a tube without being damaged, after which it can be easily positioned.

Consequently, the above-mentioned objective is achieved.

The nonwoven could be manufactured by means of a rotary-spinning process in which some of the fibers of the nonwoven are twisted together. During the rotary-spinning process, strands of fiber are formed that consist of fibers twisted together. As a result, the nonwoven acquires a particularly high level of extensibility and stability. Nonwovens of this type and methods for the production of such nonwovens are disclosed in German patent applications DE 10 2005 048 939 A1, DE 10 2007 011 606 A1, DE 10 2007 044 648 A1, European patent application EP 2 042 199 A2 and German document DE 10 2010 012 845.7, each of which is incorporated by reference herein in its entirety.

After the nonwoven has been exposed to a liquid or when it is wet, the nonwoven is still so stable that it can be sewed, glued or affixed in a similar manner. As a result, the nonwoven can be used inside the body of a human or animal without any problem.

Before this backdrop, when the nonwoven is wet or moist, it could be stretched by at least 30%, preferably by at least 70%, of its original length or original width in the unstressed state without being damaged. Consequently, the nonwoven can be sewed, glued or affixed in a similar manner inside the body of a human or animal without any problem. The nonwoven is bioresorbable in the body of humans or animals. Therefore, the nonwoven can be placed onto a wound and can then grow together with the human or animal tissue without any problem or else it can be resorbed by it.

The plunger could be configured as a medical instrument, preferably as a surgical retractor. In this context, it is advantageous that the instrument, which is used, for instance, for surgical procedures, can be employed at the same time to place the nonwoven.

At least one component of the nonwoven could contain an active ingredient or could consist of an active ingredient. In this manner, active ingredients in the form of fibers could be administered to a human or animal. It is conceivable to produce nonwovens into whose fibers active ingredients have been integrated.

At least one component could have a substance whose structure is destroyed after being heated to a temperature of at least 50° C. for at least two minutes. The term destruction of the structure here also refers to a reduction of the specific action of the substance. Such a substance can be configured as a drug, especially as an antibiotic, an enzyme, a growth factor or an analgesic.

At least one component could contain an antibiotic. Antibiotics suppress the growth of bacteria or germs.

At least one component could contain an enzyme. Enzymes can regulate metabolic processes.

At least one component could contain a growth factor. Growth factors can influence cell growth.

At least one component could contain an analgesic. As a result, the nonwovens can be placed onto wounds and can alleviate pain in the wound.

The nonwoven can consist of one or more layers. The layer or layers could be made of the polymers or polymer mixtures cited below.

Synthetic bioresorbable polymers such as polylactides, polylactide-co-glycolide copolymeres, e.g. Resomer RG 502 H, polylactide-block-polyethylene oxides, e.g. Resomer RGP d 5055, polycaprolactones, polycaprolactone-block-polyethylene oxides, polyanhydrides, e.g. polifeprosanes, polyorthoesters, polydioxanones, polyphosphoesters, for example, polylactophates, synthetic biocompatible polymers or polymers that are employed in medicine such as polyethylene glycols, polyethylene oxides, polyvinyl pyrrolidones, polyvinyl alkocols, polyethylenes, polypropylenes, polyurethanes, polydimethyl siloxanes, polymethyl methacrylates, polyvinyl chlorides, polyethylene terephthalates, polytetrafluoroethylenes, poly-2-hydroxy ethyl methacrylates, natural biopolymers such as proteins and peptides, polysaccharides, lipides, nucleic acids and especially gelatins, collagens, alginates, celluloses, elastins, starches, chitins, chitosans, hyaluronic acid, dextrans, shellack, polymer-active ingredient conjugates, namely, an active ingredient or additive bound to a bioresorbable or biocompatible polymer, as well as copolymers of the above-mentioned polymer classes.

The active ingredients cited below can be admixed to the nonwovens.

Here enzymes, antimicrobial agents, vitamins, antioxidants, anti-infectives, antibiotics, antiseptics, antiviral active ingredients, anti-rejection agents, analgesics, analgesic combinations, anti-inflammatory agents, cicatrizing agents, hormones, steroids, testosterone, estradiol, peptides and/or peptide sequences, immobilized adhesion-promoting peptide sequences, such as peptide sequences and peptide fragments of extracellular matrix proteins, especially peptides which contain one or more of the amino acid sequences RGD-, LDV-, GFOGER-, IKVAV-, SWYGLR-, COMP-, ADAM-, POEM-, YIGSR-, GVKGDKGNPGWPGAP-, cyclo-DfKRG-, KRSR-, isolated and/or genetically produced proteins, polysaccharides, glycoproteins, lipoproteins, amino acids, growth factors, particularly from the growth factor families TGF, (especially TGF-β), FGF, PDGF, EGF, GMCSF, VEGF, IGF, HGF, IL-1B, 1L8 and NG, RNA siRNA, mRNA and/or DNA, or biological signal molecules such as, for instance, Sonic Hedgelog, anticancer agents, such as paclitaxel, doxorubicin, 1,3-bis-2-chloroethyl-1-nitrosourea BCNU, camphothecin, living cells, opiates, nicotine, nitroglycerine, clonidine, fentanyl, scopolamine, rapamycine, sirolimus, gentamicin sulfate, gentamicin crobefat, aminosulfonic acids, sulfonamide peptides, peptide-analog molecules on the basis of D-amino acids, furanone derivates, dexamethasone, β-tricaldumphosphate and/or hydroxylapatite, very especially hydroxylapatite nanoparticles in concentrations ranging from 0.000001% to 70% can all be employed.

A medical instrument of the type described here could be employed in a trocar. For this purpose, the tube with the plunger, whereby the nonwoven is accommodated in the tube, can be inserted into a trocar. The trocar itself can then be inserted through a body opening.

A medical instrument of the type described here could be used to transport a nonwoven that is configured as a hemostatic nonwoven. Advantageously, the nonwoven is employed here to stanch bleeding.

A medical instrument of the type described here could be used to perform an adhesion prophylaxis. Advantageously, the nonwoven is employed here to prevent adhesions from forming between tissues.

A medical instrument of the type described here could be used to transport a nonwoven that is configured as a carrier material for bioactive substances and that is employed in local therapy. Advantageously, bioactive substances, namely, active ingredients, drugs or similar substances, can be brought via the nonwoven to their target location inside the body of a human or animal.

Nonwovens as disclosed in German patent applications DE 10 2005 048 939 A1, DE 10 2007 011 606 A1, DE 10 2007 044 648 A1, European patent application EP 2 042 199 A2 and German document DE 10 2010 012 845.7 can all be employed in the medical instrument described here, especially in a trocar.

Various possibilities exist to configure the teaching of the present invention in an advantageous manner as well as to refine it. For this purpose, reference is hereby made, on the one hand, to the subordinate claims and, on the other hand, to the explanation below of preferred embodiments of the medical instrument and nonwoven according to the invention.

Generally preferred configurations and refinements of the teaching will also be explained in conjunction with the explanation of the preferred embodiments.

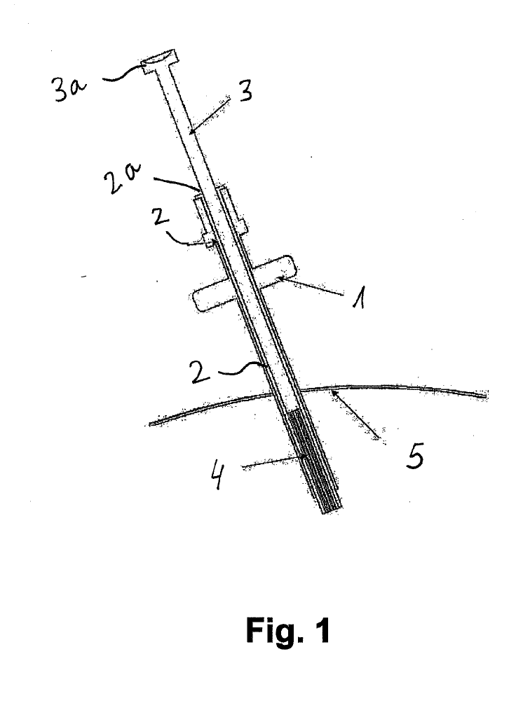

FIG. 1 shows a trocar 1 in which a medical instrument has been accommodated. FIG. 1 shows a medical instrument comprising a tube 2 and a plunger 3 accommodated inside the tube 2, whereby the plunger 3 can move relative to the tube 2 and can be slid inside it, and whereby the plunger 3 closes off a first end 2a of the tube 2. The first end 2a can also be closed off at least partially by a hollow plunger 3.

A bioresorbable nonwoven 4 is accommodated in the tube 2, whereby the nonwoven 4 can be present folded or rolled-up, and whereby the nonwoven 4 can be expelled from the tube 2 by actuating the plunger 3.

The nonwoven 4 is manufactured by means of a rotary-spinning process in which some of the fibers of the nonwoven 4 are twisted together. Nonwovens 4 of this type are disclosed in German patent applications DE 10 2005 048 939 A1, DE 10 2007 011 606 A1, DE 10 2007 044 648 A1, European patent application EP 2 042 199 A2 and German document DE 10 2010 012 845.7.

The nonwoven 4 accommodated in the tube 2 is bioresorbable in the body of humans or animals.

After the nonwoven 4 has been exposed to a fluid or when it is wet, it is so stable that it can be sewed, glued or affixed in a similar manner.

When the nonwoven 4 is wet or moist, it can be stretched by at least 30%, preferably by at least 70%, of its original length or original width in the unstressed state without being damaged.

The plunger 3 is accommodated concentrically in the tube 2 and it has an actuation disk 3a with which a thumb or finger can be used to actuate the plunger 3.

The tube 2, in turn, is accommodated concentrically in a trocar 1, whereby the nonwoven 4 is accommodated inside the tube 2 and whereby the plunger 3 can be slid inside the tube 2. The trocar 1 is inserted through the abdominal wall 5 of the body of a human.

In FIG. 1, the nonwoven 4 is still accommodated largely inside the tube 2 or inside the trocar 1.

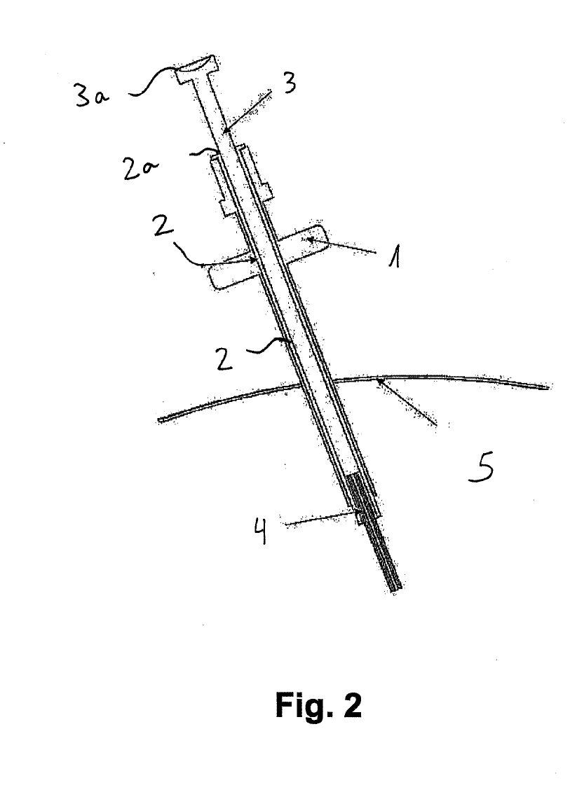

FIG. 2 shows a situation in which the plunger 3 has pushed the nonwoven 4 more than halfway out of the tube 2 or of the trocar 1.

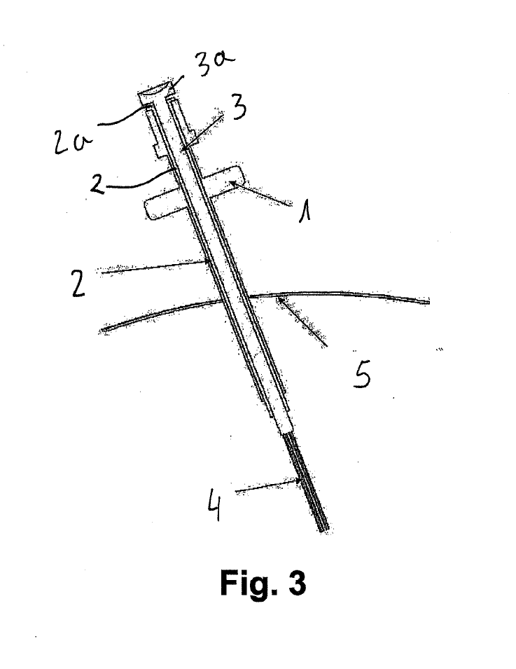

FIG. 3 shows a situation in which the plunger 3 has pushed the nonwoven 4 completely out of the tube 2 or of the trocar 1.

The nonwoven 4 can now be unfolded or unrolled and then placed onto an organ or a bone.

Concrete embodiments of the medical instrument in combination with several variants of the nonwoven 4 are presented below.

The nonwovens 4 concretely shown below stand out for the above-mentioned stability and extensibility in their wet state.

### Embodiment 1

The medical instrument schematically depicted in FIGS. 1 to 3 concretely consists of a tube 2 having an outer diameter of 9.8 mm, an inner diameter of 8 mm and a length of 210 mm. The medical instrument has a plunger 3 having a diameter of 7.95 mm and a length of 215 mm. A nonwoven 4 having a surface area of 50 mm×50 mm and a mass per unit area of 150 g/m² is accommodated in the tube 2. The tube 2 and the plunger 3 are made of polypropylene (PP).

An integral part of the medical instrument is a suitable nonwoven 4 which is employed as a three-dimensional structure to close internal wounds or to fill up defects. This nonwoven 4 is manufactured by means of a rotary-spinning process as described below.

For purposes of producing the nonwoven 4, first of all a 20%-gelatin solution is prepared. A gelatin of the type A PIGSKIN made by the GELITA AG company is used. The gelatin is stirred into water. This gelatin solution is left standing for one hour in order to swell. Subsequently, the gelatin solution is dissolved at 60° C. in an ultrasound bath and then kept at a temperature between 80° C. and 85° C. for about two hours.

This gelatin solution, which is kept at a temperature of 80° C. to 85° C., is conveyed into a container by means of a peristaltic pump as described in German patent application DE 10 2005 048 939 A1. The container is at a temperature of about 120° C. and rotates at 4500 rpm. The centripetal force causes the fiber raw material to be pressed out of the cutouts that are present in the container and this raw material is then spun into fibers. The fibers are drawn through a suction device that is located below the container.

An effective, bioresorbable nonwoven 4 made of gelatin and having an average fiber diameter of approximately 12 μm is obtained. This gelatin nonwoven is cross-linked either during the spinning process or else subsequently. A subsequent cross-linking can take place, for example, by means of a treatment with aldehydes such as, for instance, formaldehyde or glutaraldehyde. In this treatment, the nonwoven 4 is stored overnight in a vacuum drying cabinet together with a bowl containing a formaldehyde solution (Sigma-Aldrich, order no. F8775). The formaldehyde solution is removed after 24 hours. The drying cabinet is evacuated for at least 72 hours and then vented. A gelatin nonwoven treated in this manner is stable in a PBS buffer for a period of several days to weeks (preferably more than 4 weeks). PBS stands for “phosphate buffered saline” (Sigma-Aldrich P55368-10PAK). This is a physiological buffer medium having a pH value of 7.4 which is employed as the simplest model for bodily fluids.

### Embodiment 2

The medical instrument schematically depicted in FIGS. 1 to 3 consists of a tube 2 having an outer diameter of 9.8 mm, an inner diameter of 8 mm and a length of 210 mm. It comprises a plunger 3 having a diameter of 7.95 mm and a length of 215 mm. A nonwoven 4 having a surface area of 50 mm×50 mm and a mass per unit area of 150 g/m² is accommodated in the tube 2. The tube 2 and the plunger 3 are made of polymethyl methacylate (PMMA).

The nonwoven 4 has an antibiotic and is manufactured by means of a rotary-spinning process as described indicated below.

For purposes of producing the nonwoven 4, first of all a 20%-gelatin solution is prepared. A gelatin of the type A PIGSKIN according to Embodiment 1 is used. The gelatin is stirred into water. This gelatin solution is left standing for one hour in order to swell. Subsequently, the gelatin solution is dissolved at 60° C. in an ultrasound bath and then kept at a temperature between 80° C. and 85° C. for about two hours.

This gelatin solution, which is kept at a temperature of 80° C. to 85° C., is conveyed into a container by means of a peristaltic pump as described in German patent application DE 10 2008 048 939 A1. Shortly before the gelatin solution enters the cutouts, an ampoule of gentamicin solution (GENTAMICIN 40 made by the HEXAL AG company) is mixed into the gelatin solution. The container is at a temperature of about 120° C. [248° F.] and rotates at 4500 rpm. The centripetal force causes the fiber raw material to be pressed out of the cutouts that are present in the container and this raw material is then spun into fibers. The fibers are drawn through a suction device that is located below the container. After the cross-linking of the gelatin, a nonwoven 4 containing an antibiotic is obtained that has an antimicrobial effect and, at the same time, is bioresorbable.

An antimcrobially effective, bioresorbable nonwoven 4 made of gelatin and having an average fiber diameter of 12 μm is obtained. This gelatin nonwoven is cross-linked either during the spinning process or else subsequently. A subsequent cross-linking can be carried out, for example, by means of a dehydrothermal treatment. In this treatment, the nonwoven 4 is treated overnight in a vacuum drying cabinet. For this purpose, the drying cabinet is first evacuated and then heated up to 140° C. The fibers stabilized in this manner swell, but they do not dissolve right away in an aqueous environment. A gelatin nonwoven treated in this manner is water-stable for a period of several hours to days (preferably more than 2 hours).

### Embodiment 3

The medical instrument schematically depicted in FIGS. 1 to 3 consists of a tube 2 having an outer diameter of 9.8 mm, an inner diameter of 8 mm and a length of 210 mm. It comprises a plunger 3 having a diameter of 7.95 mm and a length of 215 mm as well as a nonwoven 4 having a surface area of 50 mm×50 mm and a mass per unit area of 150 g/m². The tube 2 and the plunger 3 are made of polyether ketone (PEK).

The nonwoven 4 is made of gelatin and hydroxylapatite with an antibiotic and is manufactured by means of a rotary-spinning process as described below.

For purposes of producing the nonwoven 4, first of all a 20%-gelatin solution is prepared. A gelatin of the type A PIGSKIN according to Embodiment 1 is used. The gelatin is stirred into water. This gelatin solution is left standing for one hour in order to swell. Subsequently, the gelatin solution is dissolved at 60° C. in an ultrasound bath; 2.5% nanoparticulate hydroxylapatite (Sigma-Aldrich, order no. 677418) is added, the solution is treated with ultrasound for half an hour and then kept at a temperature between 80° C. and 85° C. for about two hours.

This gelatin solution, which is kept at a temperature of 80° C. to 85° C., is conveyed into a container by means of a peristaltic pump as described in German patent application DE 10 2008 048 939 A1. The container is at a temperature of about 120° C. and rotates at 4500 rpm. The centripetal force causes the fiber raw material to be pressed out of the cutouts that are present in the container and this raw material is then spun into fibers. The fibers are drawn through a suction device that is located below the container. The cross-linking is carried out according to one of the methods described in Embodiments 1 and 2. After the gelatin has been cross-linked, the nonwoven 4 is sprayed with a solution of gentamicin and subsequently dried.

Regarding other advantageous embodiments and refinements of the teaching according to the invention, reference is hereby made, on the one hand, to the general part of the description and, on the other hand, to the accompanying patent claims.

While the invention has been particularly shown and described with reference to preferred embodiments thereof, it will be understood by those skilled in the art that various changes in form and details may be made therein without departing from the spirit and scope of the invention.

What is claimed is:

1. A medical instrument comprising:
a tube;
a bioresorbable nonwoven disposed in the tube; and
a plunger disposed inside the tube and closing off a first end of the tube, the plunger being movable relative to the tube and slidable inside the tube so as to expel the nonwoven from the tube through actuation of the plunger.

2. The medical instrument recited in claim 1, wherein the nonwoven is manufactured by a rotary-spinning process.

3. The medical instrument recited in claim 1, wherein the nonwoven is configured to have a stability when exposed to liquid or when wet to allow the nonwoven to be sewn, glued or affixed.

4. The medical instrument as recited in claim 1, wherein the nonwoven, when wet or moist is stretchable by 30% without being damaged from an original length or width in an unstressed state.

5. The medical instrument as recited in claim 1, wherein the nonwoven, when wet or moist is stretchable by 70% without being damaged from an original length or width in an unstressed state.

6. The medical instrument as recited in claim 1, wherein the plunger is a medical instrument.

7. The medical instrument as recited in claim 6, wherein the plunger is a surgical retractor.

8. A method of using a medical instrument comprising:
providing a medical instrument including:
a tube,
a bioresorbable nonwoven disposed in the tube, and
a plunger disposed inside the tube and closing off a first end of the tube, the plunger being movable relative to the tube and slidable inside the tube;
disposing the medical instrument in a trocar; and
actuating the plunger so as to expel the nonwoven from the tube.

9. A method of using a medical instrument comprising:
providing a medical instrument including:
a tube,
a bioresorbable nonwoven disposed in the tube, and
a plunger disposed inside the tube and closing off a first end of the tube, the plunger being movable relative to the tube and slidable inside the tube; and
actuating the plunger so as to expel the nonwoven from the tube.

10. The method recited in claim 9, wherein the nonwoven is a hemostatic nonwoven.

11. The method recited in claim 9, wherein the actuation of the plunger so as to expel the nonwoven from the tube is carried out so as to perform an adhesion prophylaxis.

12. The method recited in claim 9, wherein the nonwoven is a carrier material for bioactive substances and is expelled from the tube so as to employ local therapy.
